(19) United States
(12) Patent Application Publication
(10) Pub. No.: US 2017/0258724 A1
(43) Pub. Date: Sep. 14, 2017

(54) METHOD OF BORON NITRIDE NANOPARTICLE FABRICATION FOR ANTITUMOR DRUG DELIVERY

(71) Applicant: National University of Science and Technology "MISiS", Moscow (RU)

(72) Inventors: Dmitry Vladimirovich Shtansky, Moscow (RU); Andrey Mikhailovich Kovalskii, Moscow (RU); Andrei Trofimovich MATVEEV, Minsk (BY); Irina Viktorovna SUKHORUKOVA, Moscow (RU); Natalia Aleksandrovna GLOUSHANKOVA, Moscow (RU); Irina Yur`evna ZHITNYAK, pos. Ganino (RU)

(21) Appl. No.: 15/600,510

(22) Filed: May 19, 2017

Related U.S. Application Data
(63) Continuation of application No. PCT/RU2015/000064, filed on Feb. 4, 2015.

(30) Foreign Application Priority Data
Nov. 20, 2014 (RU) 2014146689

(51) Int. Cl. A61K 9/14; A61K 31/704

(52) U.S. Cl. CPC A61K 9/143; A61K 31/704

(57) ABSTRACT

This invention relates to nanomedical technologies, namely to the fabrication of nanocarriers of drugs for antitumor chemotherapy. The technical result of the invention consists of increasing the efficiency of antitumor chemotherapy by increasing the activity of the cell absorption of nanocontainers loaded with an antitumor drug, avoiding of the nanocontainer toxicity for cells due to the use of dispersed boron nitride nanoparticles of 50-300 nm in diameter with a well-developed outer surface. The method of boron nitride nanoparticles fabrication for antitumor drug delivery to tumor cells includes synthesis of spherical boron nitride nanoparticles of 50-300 nm in diameter with a well-developed outer surface by chemical vapor deposition using ammonia reaction gas, argon transport gas and powder mixture composed of amorphous boron and oxidizing reactants.

### FIELD OF THE INVENTION

This invention relates to nanomedical technologies, more specifically, to the development of transporting nanoagents for the delivery of antitumor chemotherapy drugs.

The reduced efficiency of antitumor chemotherapy during the treatment of oncology patients is primarily attributed to the progression of malignant neoplasms and the generation of cells resulting from tumor progression that have the so-called multiple drug resistance caused by the activation of membrane transporting agents that remove antitumor drugs from the cells. Furthermore, a number of drugs used for the therapy of oncological diseases are low hydrophilic ones and have a toxic effect on healthy body tissues. One way to solve these problems is to use nanotransporting agents delivering drugs to tumor cells.

### PRIOR ART

Known is a method of obtaining spherical boron nitride nanoparticles by chemical vapor deposition (US 20110033707, published Feb. 10, 2011). This method allows to obtain boron nitride nanoparticles less than 50 nm in diameter. The method includes heating a mixture of an ether, boric acid and nitrogen in an ammonium and argon atmosphere leading to the formation of the reaction product, its further crystallization which results in formation of spherical nanoparticles precursor and final heating of the precursor in an inert gas.

A disadvantage of this method is the small size of the particles and their smooth surface which does not allow loading the drug in amount required for efficient antitumor therapy and limits the attachment of these particles to tumor cells and their penetration into the latter.

Known is a method of obtaining nanoparticles for drug delivery (WO 2014124329, published Aug. 14, 2014).

This method uses spherical magnetic particles of iron or iron oxide including those coated with gold or silicon oxide. For the therapy of oncological diseases the drug is attached to the magnetic particles through the use of a linker, e.g. ether. The particles heat up by the means of external heating or electromagnetic field, causing the intramolecular cyclization of the linker which results in a release of the drug. This method allows controlled release of drugs thus minimizing side effects and increasing the therapeutic efficiency of usual drugs.

A disadvantage of this method is the necessity of using additional external effect for achieving the required therapeutic efficiency. Another drawback of this method is that some forms of magnetic particles may give rise to the toxicity.

Known is a method for manufacturing nano-carriers, complex of anticancer drug and nano-carrier and pharmaceutical composition for the therapy of oncological diseases (US 20140147508, published May 29, 2014).

This invention relates to a nano-carrier for an anticancer drug, comprising of a spherical metallic nanoparticle and a polynucleotide for drug attachment. The method allows providing a therapeutic effect on tumor cells through the controlled release of a drug in the affected area.

A disadvantage of this method is that the nano-carrier do not deliver drug directly into the tumor cells, so the drug release is carried out in the vicinity of the cells. This reduces the efficiency of the method due to the development of multi drug resistance in the tumor cells.

Also known is a method of delivering anticancer drug to a cancer cells with metal nanoparticles (US 20130331764, published Dec. 12, 2013). The antitumor drug delivery is carried out by the pH-sensitive gold particles of 5-15 nm. After entering the body these pH-sensitive metal nanoparticles are dispersed in normal cells, which are neutral or alkaline, and selectively aggregate in cancer cells which have acidic pH. The therapeutic effect of this method is achieved through the chemical effect of the antitumor drug and the photothermal effect of particle aggregates heated by NIR radiation which deeply penetrates into the patient's body.

A disadvantage of this method is the necessity of additional IR irradiation and the absence of therapeutic efficiency of drug penetration without additional external effect on the patient's body. Furthermore, particles aggregate in tumor cells selectively and thus method efficiency cannot be predicted. Also the 5-15 nm particle size is not desirable due to its excessively rapid absorption by the reticuloendothelial system.

As a prototype we selected a method of obtaining boron nitride nanotubes functionalized with mesoporous silica for intracellular delivery of chemotherapy drugs (X. Li, Ch. Zhi, N. Hanagata, M. Yamaguchi, Y. Bando and D. Golberg, Boron Nitride Nanotubes Functionalized with Mesoporous Silica for Intracellular Delivery of Chemotherapy Drugs, Chem. Commun., 2013, 49, 7337). Method implies obtaining boron nitride nanotubes by chemical vapor deposition, their oxidation in air for 5 h, formation of a mesoporous silica coating by means of TEOS hydrolysis and further saturation with an antitumor drug. This method allows to increase the efficiency of doxorubicin antitumor drug by 3-4 times compared with free doxorubicin.

The main disadvantage of this method is the unfavorable morphology of boron nitride for cell absorption. Nanotube shaped particles are known to have insufficient activity of cellular uptake and they may be toxic.

### DISCLOSURE OF THE INVENTION

The technical result achieved in the invention is that increasing the efficiency of antitumor chemotherapy by increasing of activity of taking up of nanocontainers with an antitumor drug by cells, prevention of the nanocontainer toxicity for cells due to application of dispersed born nitride nanoparticles 50-300 nm in size with a well-developed outer surface.

Said technical result is achieved in a certain way.

Method of boron nitride nanoparticles fabrication for antitumor drug delivery to tumor cells involves synthesis of spherical boron nitride nanoparticles 50-300 nm in size with a well-developed outer surface by chemical vapor deposition method using reacting ammonia gas, transport argon gas and powder mixtures on the base of amorphous boron and oxygen carrier chemicals.

Chemical deposition is carried out under the following conditions.

1000≦T≦1430

1.2≦ξ≦8,

where T is the temperature of powder mixture, ξ is the ratio of the specific flows F_{Ar}/F_{NH3}, wherein F_{Ar} is a specific flow of transport gas and F_{NH3} is a specific flow of reacting gas.

Then using ultrasonication the agglomerates of obtained boron nitride nanoparticles are dispersed, saturated with an antitumor drug by sorption and washed with distilled water.

Boric acid and/or magnesium oxide and/or iron oxide (II) and/or tin oxide (II) and/or their mixtures can be used as an oxygen carrier chemicals.

The content of iron oxide and amorphous boron in the powder mixture is taken based on the following weight ratio, wt. %:
iron oxide 70-91
amorphous boron 9-30

The content of magnesium oxide and amorphous boron in the powder mixture is taken based on the following weight ratio, wt. %:
magnesium oxide 65-84
amorphous boron 16-35

The content of tin oxide and amorphous boron in the powder mixture is taken based on the following weight ratio, wt. %:
tin oxide 75-95
amorphous boron 5-25

The content of boric acid and amorphous boron in the powder mixture is taken based on the following weight ratio, wt. %:
boric acid 85-92%
amorphous boron 8-15%

The content of iron oxide, magnesium oxide and amorphous boron in the powder mixture is taken based on the following weight ratio, wt. %:
iron oxide 59-86
magnesium oxide 5-12
amorphous boron 9-32

The content of boric acid, magnesium oxide and amorphous boron in the powder mixture is taken based on the following weight ratio, wt. %:
boric acid 65-91
magnesium oxide 3-10
amorphous boron 6-25

Boron nitride nanoparticles are dispersed using ultrasound method with a power of 40-100 W for 30 minutes.

Sorption of an antitumor drug on boron nitride nanoparticles is perform by continuous agitation of the dispersed nanoparticles in an antitumor drug solution with a concentration of 0.5-5.0 mg/ml for 12-24 h using a magnetic stirrer at a 250 rpm speed.

Sorption of an antitumor drug on boron nitride nanoparticles alternatively can be carried out using ultrasonic treatment of the dispersed nanoparticles in an antitumor drug solution with a concentration of 0.5-5.0 mg/ml with power of 150 W for 15-60 min.

Said antitumor drug is selected from synthetic or natural antitumor drugs.

Said synthetic antitumor drug is selected from alkylating drugs or metabolic antagonists or synthetic drugs of other groups.

Said alkylating synthetic antitumor drug is selected from chloroethylamines or ethyleneamines or nitrosourea derivatives or methanesulfonic acid derivatives.

Said metabolic antagonist synthetic antitumor drug is selected from folic acid antagonists or purine antagonists or pyrimidine antagonists.

Said synthetic antitumor drug of other groups is selected from prospidinum or spirasidine or dicarbasine or natulan or cisplatine or imizadolecarboxamide.

Said natural antitumor drug is selected from antibiotics or phytogenous drugs.

Said natural antitumor drug of the antibiotics group is selected from adriamycin or bleomycin or dactinomycin or rubomycin or bruneomycin or mitomycin C.

Said natural antitumor drug of vegetable origin is selected from colchamine or vinblastine or vincristine.

The boron nitride nanoparticles with a well-developed outer surface are characterized by a hollow spherical central part and multiple petals of exposed BN layers at the side area. The high porosity and the significantly increased specific surface area of the nanoparticles allow boron nitride nanocontainers to retain medical drug molecules and deliver them to tumor cells thus increasing the efficiency of antitumor therapy.

### EMBODIMENTS OF THE INVENTION

The invention is implemented as follows.

A powder mixture comprising oxidizing reactants and amorphous boron is prepared. The following reactants are used: boric acid and/or magnesium oxide and/or iron oxide (II) and/or tin oxide (II) and/or mixtures thereof.

The magnesium oxide powder is preliminarily annealed in a muffle or tube electric furnace in air at 400° C.

The powder mixture components are weighed on a laboratory balance with an accuracy of 0.1 mg.

Synthesis of spherical boron nitride nanoparticles is carried out using a high-temperature induction furnace. The crucible containing the powder mixture is placed onto the bottom of the reactor made from boron nitride which is installed inside a graphite heater in the reactor chamber.

The reactor is a demountable cylindrical structure with an outer diameter of 75 mm, an inner diameter of 55 mm and a height of 340 mm. The side wall of the reactor has a 3 mm diameter opening in its upper section for the release of synthesized spherical boron nitride nanoparticles. The graphite heater of the reactor is spaced from the reactor chamber walls with carbon/carbon composite lining The reactor design provides delivery of the argon transport gas from the bottom upwards and the ammonium reactant gas from the top downwards.

The size and chemical composition of the particles are controlled using a JEOL JSM-7600F electron microscope (JEOL, Japan) equipped with an energy-dispersive X-ray spectroscopy detector.

The average size of the spherical nanoparticles varies between 50 and 300 nm and primarily depends on the synthesis temperature and the flow rates of the transport gas (argon) and the reactant gas (ammonia).

The experimental temperature determines the volume fraction of boron nitride nanoparticles in the synthesis products and their morphology. To obtain boron nitride nanoparticles of the required size and morphology in a quantity of above 80% (by volume) one should carry out the synthesis at temperatures of 1000-1430° C. on powder mixture. Below 1000° C. the growth rate of boron nitride structures significantly slows down due to the low reaction speed between boron and the oxidizing reactant, while above 1430° C. a volume fraction of spherical boron nitride nanoparticles of the required size is decreasing.

The reaction between amorphous boron and the oxidizing reactant produces volatile boron oxides which are carried over by the transport gas (argon) to the ammonia reaction area where the nanostructures synthesis takes place.

The ratio of the flows of argon transport gas and ammonia reaction gas determines the kinetics of boron nitride nanoparticle synthesis and hence the diameter of the nanostructures. High volume fractions of 50-300 nm sized nanoparticles (>80 vol. %) are achieved at the flow ratio ξ=F_{Ar}/F_{NH3} in the range 1.2≦ξ≦8.

At gas flow ratios ξ≦1.2 the ammonia reaches the powder mixture and produces a layer of boron nitride microparticles on its surface which prevents further release of gaseous boron oxide and thus significantly impedes the synthesis of spherical boron nitride nanoparticles with a resultant abrupt decline in a final product output.

At gas flow ratios ξ>8 gaseous boron oxide has no enough time for complete reaction with ammonia which also reduces the output of spherical boron nitride nanoparticles and causes the formation of large quantities of solid boron oxide.

Ratio of components in the powder mixture is selected from the abovementioned ranges.

Beyond those ranges, either no spherical boron nitride nanoparticles form, or their size changes, or the quantity of spherical boron nitride nanoparticles of the required size in synthesis products decreases.

Spherical boron nitride nanoparticles with the well-developed outer surface form in a result of the interaction of boron oxide vapors and ammonia within the limited temperature range of (1000-1430° C.) at a specific flow ratio of the argon transport gas carrying gaseous boron oxide and the ammonia reaction gas during synthesis.

Spherical boron nitride nanoparticles are often agglomerated in synthesis products. To obtain suspension containing individual boron nitride nanoparticles, one should carry out ultrasonic dispersion at a power of 40-100 W for 30 min in 40 ml of distilled water. The range of ultrasound power of the 40-100 W is chosen because at lower powers the boron nitride nanoparticle agglomerations do not split into individual nanoparticles, whereas higher power causes heating of the suspension which may lead to partial hydrolysis of BN nanoparticles. If ultrasonic treatment is carried out less than 30 minutes the required agglomeration dispersion degree is not achieved, while treatment longer than 30 minutes may destruct the nanoparticles.

For making boron nitride nanoparticles capable of destroying tumor cells they are saturated with an antitumor drug.

Saturation of boron nitride nanoparticles with an antitumor drug is achieved by sorption through continuous agitation of the dispersed nanoparticles in an antitumor drug solution with a concentration of 0.5-5.0 mg/l for 12-24 h using a Heidolph MR (Hei-Standard, Germany) magnetic stirrer at a speed of 250 rpm. Concentrations less than 0.5 mg/ml do not ensure the required therapy efficiency, while at concentrations of greater than 5.0 mg/ml the suspension of BN nanoparticles does not retain stability for more than 24 h.

The time required for the boron nitride nanoparticles saturation with an antitumor drug is at least 12 h. Treatment for less than 12 h does not achieve the limit saturation of the nanoparticles with the drug. Treatment for 12-24 h is sufficient for the limit saturation of the nanoparticle surface with the antitumor drug. Treatment for more than 24 h does not provide any additional sorption of the antitumor drug by the nanoparticles.

An alternative method of saturating the particles with the drug is ultrasonic treatment on a Bandelin Sonoplus HD2200 device, Germany, at 150 W for 15-60 min.

Higher power and longer treatment may destruct the molecules of the antitumor drug, while below these ranges the treatment will not provide the necessary concentration of antitumor drug required in the boron nitride nanoparticles.

Studies of the biocompatibility of boron nitride nanoparticles have shown that the 50-300 nm nanoparticle size is the optimum one from the viewpoint of their absorption by tumor cells. Furthermore the surface of finer particles does not provide for the transportation of the sufficient quantity of drug for efficient destruction of tumor cells, whereas larger particles are not consumed by tumor cells.

Spherical boron nitride nanoparticles with a well-developed outer surface provide the required sorption value of the drug and its subsequent delivery to the tumor cells.

Table 1 shows embodiments of the invention with different synthesis parameters of boron nitride nanoparticles with a well-developed outer surface. Table 2 shows embodiments of the invention with different antitumor drug saturation methods.

### EXAMPLE 1

Spherical boron nitride nanoparticles were synthesized by chemical vapor deposition in a vertical induction furnace VIN-1,6-20 (Vac ETO Co., Russia). A crucible with thoroughly homogenized powder mixture (mass was 10.88 g) consist of 59 wt. % iron oxide, 12 wt. % magnesium oxide and 29 wt. % amorphous boron was placed in the furnace. The working chamber of the reactor was heated to 300° C. assisted by permanent vacuum pumping for removal of water and oxygen adsorbed on the work chamber surface. Then the chamber was filled with argon to atmospheric pressure. Flows of argon transport gas and ammonia reaction gas were open in the furnace after heating to 850° C. After heating to 1310° C., the temperature was kept constant during 200 min. The working chamber was cooled down to 40-50° C. after the synthesis final followed by ventilation with air and opening.

The snow-white fozy powder with masse of 280 mg was received as results of synthesis. According to SEM observations, this snow powder is revealed of agglomerates of boron nitride nanoparticles of 1-5 μm in diameter. Individual BN nanoparticles with diameter of 100-150 nm had a spherical shape and well-developed outer surface.

Stable suspension of individual boron nitride nanoparticles with a well-developed outer surface was obtained in the ultrasonic disperser “Sonoplus HD2200” (Bandelin, Germany). Ultrasonic treatment during 30 minutes in a 40 ml of distilled water at disperser power of 100 W was carried out due to separate the BN nanoparticles agglomerates. The boron nitride nanoparticles concentration was 2 mg/ml. Study of the particle size distribution after ultrasonic treatment showed that more than 80% of the particles have diameter of 100-200 nm.

Spherical boron nitride nanoparticles were saturated with an antitumor drug by continuous mixing of boron nitride nanoparticles in an antitumor drug solution in a magnetic stirrer “Heidolph MR” (Hei-Standard, Germany) Nanoparticles were separated from the liquid phase using an Eppendorf (Germany) centrifuge. The concentration of the doxorubicin antitumor drug water solution was 0.5 mg/ml. After antitumor drug saturation, boron nitride nanoparticles were triple washed in distilled water and centrifuged for the removal of drug residues from nanoparticles surface.

The absorption of drug saturated nanoparticles by neoplasm cells was studied using confocal microscopy of a culture of Alexa488-falloidine colored cells. The viability of the neoplasm cells after exposure with nanoparticles was studied by measuring the dynamics of transformed cell proliferation during cultivation in the growth media in the presence of drug saturated nanoparticles during 7 days and comparing the results with control samples using fluorescent microscopy control and DAPI staining.

Confocal microscopy showed that boron nitride nanoparticles loaded with doxorubicin are capable to penetrate into transformed (tumor) cells of the IAR-6-1 line. The viability test results showed that inside of tumor cells doxorubicin is released from particles and destructs the cell.

### EXAMPLE 2

Powder mixture with mass of 11.76 g consist of 78 wt. % boric acid, 4 wt. % magnesium oxide and 18 wt. % amorphous boron was placed in a crucible in the reactor. After preliminary reactor purification from impurities, inert gas filling, heating to the working temperature and the delivery of the transport and reactant gas flows (methodic described in Example 1), the work temperature was established at 1190° C. and kept constant during 320 min. The result of synthesis was snow-white fozy powder with mass of 345 mg that were agglomerated of BN nanoparticles with diameter 70-100 nm.

Ultrasonic dispersion was carried out in distilled water at a power of 80 W during 30 min. The concentration of boron nitride nanoparticles was 2 mg/ml. Study of the particle size distribution showed that the quantity of nanoparticles and their agglomerates greater than 250 nm in size was less than 1%.

Boron nitride nanoparticles were saturated with an antitumor drug by ultrasonic treatment of nanoparticles in a 5 mg/ml doxorubicin water solution during 15 min.

Biological tests using transformed (tumor) IAR-6-1 cells in accordance with methodic described in Example 1, showed that the cells successfully absorbed doxorubicin loaded nanoparticles that caused cell death.

TABLE 1
Synthesis Parameters of BN Nanoparticles with a Well-Developed Outer Surface

| | | | | | | | | | Diameter of | Volume fraction |
|---|---|---|---|---|---|---|---|---|---|---|
| | | | | | | | | Spherical BN | spherical | of BN |
| | Powder mixture composition, | | | | | | | nanoparticles with a | BN | nanoparticles in |
| | wt. % | | | | | | | well-developed | nanoparticles, | synthesis |
| No | FeOx | MgO | H₃BO₃ | SnO | B | T, ° C. | ξ | outer surface | nm | products, % |

Optimal parameters corresponding to the claimed formula

| | | | | | | | | | | |
|---|---|---|---|---|---|---|---|---|---|---|
| 1 | 59 | 12 | — | — | 29 | 1310 | 2 | + | 80-100 | >80 |
| 2 | 65 | 3 | — | — | 32 | 1200 | 1.2 | + | 100-120 | >80 |
| 3 | 86 | 5 | — | — | 9 | 1130 | 6 | + | 100-300 | >80 |
| 4 | — | 10 | 65 | — | 25 | 1410 | 2 | + | 100-120 | >80 |
| 5 | — | 5 | 70 | — | 25 | 1410 | 8 | + | 100-120 | >80 |
| 6 | — | 4 | 78 | — | 18 | 1190 | 6 | + | 50-100 | >80 |
| 7 | — | 3 | 91 | — | 6 | 1310 | 4 | + | 100-110 | >80 |
| 8 | 70 | — | — | — | 30 | 1320 | 2 | + | 100-140 | >80 |
| 9 | 83 | — | — | — | 17 | 1205 | 1.5 | + | 100-200 | >80 |
| 10 | 91 | — | — | — | 9 | 1000 | 2 | + | 150-300 | >80 |
| 11 | — | 65 | — | — | 35 | 1350 | 6 | + | 100-120 | >80 |
| 12 | — | 71 | — | — | 29 | 1430 | 6 | + | 50-100 | >80 |
| 13 | — | 84 | — | — | 16 | 1430 | 2 | + | 80-100 | >80 |
| 14 | — | — | — | 95 | 5 | 1320 | 4 | + | 100-120 | >80 |
| 15 | — | — | — | 87 | 13 | 1245 | 2 | + | 50-200 | >80 |
| 16 | — | — | — | 75 | 25 | 1410 | 4 | + | 90-110 | >80 |
| 17 | — | 40 | — | — | 60 | 1430 | 2 | + | 90-110 | >80 |
| 18 | — | — | 85 | — | 15 | 1270 | 1.5 | + | 100-120 | >80 |
| 19 | — | — | 89 | — | 11 | 1330 | 2 | + | 100-120 | >80 |
| 20 | — | — | 92 | — | 8 | 1000 | 1.2 | + | 160-200 | >80 |

TABLE 2
Modes of Nanoparticle Saturation with Antitumor Drug

| | | | | | | | |
|---|---|---|---|---|---|---|---|
| | Method of BN | | Antitumor drug | | | | |
| | nanoparticle | | solution | | | Nanoparticle | |
| | saturation with | Ultrasonic | concentration, | Treatment | Distilled water | absorption | |
| No | antitumor drug | dispersion | mg/ml | time, h | washing | by cells | Therapy effect |

Optimal parameters corresponding to the claimed formula

| | | | | | | | |
|---|---|---|---|---|---|---|---|
| 2 | Ultrasonic | + | 0.5 | 0.75 | + | + | + |
| | treatment | | | | | | |
| 6 | Ultrasonic | + | 5 | 0.25 | + | + | + |
| | treatment | | | | | | |
| 6 | Ultrasonic | + | 2 | 0.25 | + | + | + |
| | treatment | | | | | | |
| 6 | Ultrasonic | + | 2 | 1 | + | + | + |
| | treatment | | | | | | |
| 1 | Continuous | + | 0.5 | 12 | + | + | + |
| | mixing with a | | | | | | |
| | magnetic stirrer | | | | | | |
| 1 | Continuous | + | 5 | 18 | + | + | + |
| | mixing with a | | | | | | |
| | magnetic stirrer | | | | | | |
| 1 | Continuous | + | 2 | 12 | + | + | + |
| | mixing with a | | | | | | |
| | magnetic stirrer | | | | | | |
| 8 | Continuous | + | 2 | 24 | + | + | + |
| | mixing with a | | | | | | |
| | magnetic stirrer | | | | | | |

What is claimed is:

1. A method of boron nitride nanoparticles fabrication for antitumor drug delivery to tumor cells involves synthesis of spherical boron nitride nanoparticles 50-300 nm in size with a well-developed outer surface by chemical vapor deposition method using reacting ammonia gas, transport argon gas and powder mixtures on the base of amorphous boron and oxygen carrier chemicals wherein chemical deposition is carried out under the following conditions:
1000≦T≦1430
1.2≦ξ≦8,
where T is the temperature of powder mixture, ξ is the ratio of the specific flows F_{Ar}/F_{NH3}, wherein F_{Ar} is a specific flow of transport gas and F_{NH3} is a specific flow of reacting gas, followed by the ultrasonic dispersion of agglomerates of obtained boron nitride nanoparticles, saturation with an antitumor drug by sorption and washing in distilled water.

2. The method of claim 1 wherein oxygen carrier chemicals can be selected from the boric acid and/or magnesium oxide and/or iron oxide (II) and/or tin oxide (II) and/or their mixtures.

3. The method of claim 2 wherein the content of iron oxide and amorphous boron in the powder mixture is taken based on the following weight ratio, wt. %:
iron oxide 70-91
amorphous boron 9-30

4. The method of claim 2 wherein the content of magnesium oxide and amorphous boron in the powder mixture is taken based on the following weight ratio, wt. %:
magnesium oxide 65-84
amorphous boron 16-35

5. The method of claim 2 wherein the content of tin oxide and amorphous boron in the powder mixture is taken based on the following weight ratio, wt. %:
tin oxide 75-95
amorphous boron 5-25

6. The method of claim 2 wherein the content of boric acid and amorphous boron in the powder mixture is taken based on the following weight ratio, wt. %:
boric acid 85-92%
amorphous boron 8-15%

7. The method of claim 2 wherein the content of iron oxide, magnesium oxide and amorphous boron in the powder mixture is taken based on the following weight ratio, wt. %:
iron oxide 59-86
magnesium oxide 5-12
amorphous boron 9-32

8. The method of claim 2 wherein the content of boric acid, magnesium oxide and amorphous boron in the powder mixture is taken based on the following weight ratio, wt. %:
boric acid 65-91
magnesium oxide 3-10
amorphous boron 6-25

9. The method of claim 1 wherein boron nitride nanoparticles are dispersed with ultrasound treatment with a power of 40-100 W for 30 minutes.

10. The method of claim 1 wherein sorption of an antitumor drug on boron nitride nanoparticles is perform by continuous agitation of the dispersed nanoparticles in an antitumor drug solution with a concentration of 0.5-5.0 mg/ml for 12-24 h using a magnetic stirrer at a 250 rpm speed.

11. The method of claim 1 wherein sorption of an antitumor drug by boron nitride nanoparticles alternatively can be carried out by ultrasonic treatment of the dispersed nanoparticles in an antitumor drug solution with a concentration of 0.5-5.0 mg/ml with a power of 150 W for 15-60 min.

12. The method of claim 1 wherein said antitumor drug is selected from synthetic or natural antitumor drugs.

13. The method of claim 12 wherein said synthetic antitumor drug is selected from alkylating drugs or metabolic antagonists or synthetic drugs of other groups.

14. The method of claim 13 wherein said alkylating synthetic antitumor drug is selected from chloroethylamines or ethyleneamines or nitrosourea derivatives or methanesulfonic acid derivatives.

15. The method of claim 13 wherein said metabolic antagonist synthetic antitumor drug is selected from folic acid antagonists or purine antagonists or pyrimidine antagonists.

16. The method of claim 13 wherein said synthetic antitumor drug of other groups is selected from prospidinum or spirasidine or dicarbasine or natulan or cisplatine or imizadolecarboxamide.

17. The method of claim 12 wherein said natural antitumor drug is selected from antibiotics or phytogenous drugs.

18. The method of claim 17 wherein said natural antitumor drug of the antibiotics group is selected from adriamycin or bleomycin or dactinomycin or rubomycin or bruneomycin or mitomycin C.

19. The method of claim 17 wherein said natural antitumor phytogenous drug is selected from colchamine or vinblastine or vincristine.
